(19) United States
(12) Patent Application Publication
(10) Pub. No.: US 2025/0017679 A1
(43) Pub. Date: Jan. 16, 2025

(54) MEDICAL SUPPLY PACKAGE

(71) Applicant: TERUMO KABUSHIKI KAISHA, Tokyo (JP)

(72) Inventors: Kiyoaki HASUMI, Toda-shi (JP); Junichi OGAWA, Kofu-shi (JP)

(73) Assignee: TERUMO KABUSHIKI KAISHA, Tokyo (JP)

(21) Appl. No.: 18/897,103

(22) Filed: Sep. 26, 2024

Related U.S. Application Data
(63) Continuation of application No. PCT/JP2022/045544, filed on Dec. 9, 2022.

(30) Foreign Application Priority Data
Mar. 28, 2022 (JP) 2022-052078

(51) Int. Cl. A61B 50/30; A61B 50/00

(52) U.S. Cl. CPC A61B 50/30; A61B 2050/005

(57) ABSTRACT

A medical supply package storing a medical supply, the medical supply package including: a first sheet member; and a second sheet member facing the first sheet member. The medical supply is stored between the first sheet member and the second sheet member. The first sheet member and/or the second sheet member comprises a storage recess having a shape corresponding to an outer shape of the medical supply in a central portion. The first sheet member and the second sheet member include: abutting portions surrounding an outer periphery of the storage recess and facing each other, an annular seal portion formed on the abutting portions and surrounding and hermetically sealing the storage recess, and a non-seal portion provided on an outer side of the annular seal portion. At least one of the first sheet member and the second sheet member comprises a linear or band-shaped breakable portion.

### CROSS-REFERENCE TO RELATED APPLICATIONS

This is a bypass continuation of PCT Application No. PCT/JP2022/045544, filed on Dec. 9, 2022, which claims priority to Japanese Patent Application No. 2022-052078, filed on Mar. 28, 2022. The entire contents of these application are incorporated herein by reference.

### BACKGROUND

The present disclosure relates to a medical supply package that stores a medical supply, the medical supply package from which the stored medical supply can be easily taken out while securing airtightness.

Conventionally, various medical supplies are distributed in a state of being stored in a package (as a medical supply package). As such a medical supply package, for example, Japanese Patent Publication No. 2007-297060 A (“Patent Literature 1”) discloses a blister package in which a lid member is sealed on a container main body provided with a housing molded in a drawn shape in such a manner that a prefilled syringe, which is a medical product, is stored.

In Patent Literature 1, an opening of a container in which a prefilled syringe is stored is sealed (sealed) by a lid member (sealing film 101) in a seal portion (22) provided on an entire peripheral edge of a container main body (21).

### SUMMARY

In a medical supply package that stores a medical supply, airtightness is required for the package to prevent outside air, foreign matter and the like from entering the package from outside, or to prevent fluid (gas or liquid (drug solution)) and an object (drug and the like) in the package from leaking to the outside. It is also important to prevent airtightness from being impaired by an impact or the like during transportation. However, if the seal is made strong in the medical supply package to satisfy such a requirement (of securing airtightness), it becomes difficult to open the package. In other words, in the medical supply package, it becomes difficult to take out the stored medical supply, and there is also a possibility that the medical supply is damaged when being taken out.

Therefore, an object of certain embodiments of the present disclosure is to provide a medical supply package that stores a medical supply, the medical supply package from which the stored medical supply can be easily taken out while securing airtightness.

The object described above is achieved by the following.

A medical supply package storing a medical supply, the medical supply package including a first sheet member and a second sheet member facing the first sheet member, the medical supply package storing the medical supply between the first sheet member and the second sheet member, in which the first sheet member and/or the second sheet member includes a storage recess having a shape corresponding to an outer shape of the medical supply in a central portion, the first sheet member and the second sheet member includes abutting portions surrounding an outer periphery of the storage recess and facing each other, an annular seal portion formed on the abutting portions facing each other, surrounding the storage recess, and hermetically sealing the storage recess, and a non-seal portion provided on an outer side of the annular seal portion, and at least one of the first sheet member and the second sheet member includes a linear or band-shaped breakable portion, the breakable portion includes an opening start portion including a start end in the non-seal portion, a peelable portion continuous from the opening start portion and crossing a part of the annular seal portion, and a breakable portion main part continuous from the peelable portion and crossing the storage recess, and the breakable portion does not inhibit airtightness of the annular seal portion.

The object described above is achieved by the following.

A medical supply package storing a medical supply, the medical supply package including a first sheet member and a second sheet member facing the first sheet member, the medical supply package storing the medical supply between the first sheet member and the second sheet member, in which the first sheet member and/or the second sheet member includes a storage recess having a shape corresponding to an outer shape of the medical supply in a central portion, the first sheet member and the second sheet member includes abutting portions surrounding an outer periphery of the storage recess and facing each other, and an annular seal portion formed on the abutting portions facing each other, surrounding the storage recess, and hermetically sealing the storage recess, at least one of the first sheet member and the second sheet member includes a breakable linear portion surrounding an inner side of the annular seal portion and the storage recess, and the medical supply package includes an opening tab one end of which is fixed to an inner edge of the breakable linear portion and the other end of which is a free end, in which the breakable linear portion is formed of intermittently formed cuts, and do not penetrate the first sheet member or the second sheet member provided with the breakable linear portion.

### BRIEF DESCRIPTION OF THE DRAWINGS

FIG. 1 is a front view illustrating an example of a medical supply package of the present invention.

FIG. 2 is a cross-sectional view taken along line A-A of FIG. 1.

FIG. 3 is a front view illustrating a first sheet member of the medical supply package illustrated in FIG. 1.

FIG. 4 is a back view illustrating the first sheet member of the medical supply package illustrated in FIG. 1.

FIG. 5 is a front view illustrating another example of the medical supply package of the present invention.

FIG. 6 is a front view illustrating another example of the medical supply package of the present invention.

FIG. 7 is a front view illustrating another example of the medical supply package of the present invention.

FIG. 8 is a front view illustrating another example of the medical supply package of the present invention.

FIG. 9 is a cross-sectional view corresponding to FIG. 2 illustrating another example of the medical supply package of the present invention.

FIG. 10 is a front view illustrating another example of the medical supply package of the present invention.

FIG. 11 is a front view illustrating another example of the medical supply package of the present invention.

FIG. 12 is a front view illustrating another example of the medical supply package of the present invention.

FIG. 13 is a front view illustrating another example of the medical supply package of the present invention.

### DETAILED DESCRIPTION

A medical supply package of the present disclosure will be described with reference to examples illustrated in the drawings.

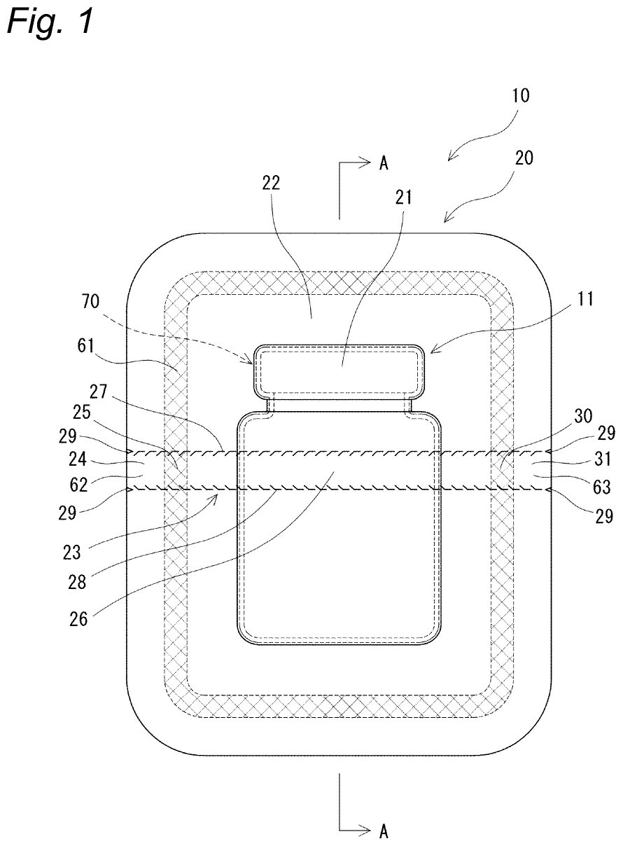

As illustrated in FIGS. 1 to 4, a medical supply package 10 of the present invention includes a first sheet member 20 and a second sheet member 40 facing the first sheet member 20, and stores a medical supply 70 between the first sheet member 20 and the second sheet member 40. The first sheet member 20 and/or the second sheet member 40 includes a storage recess 11 (first sheet-side recess (first storage recess) 21 and a second sheet-side recess (second storage recess) 41) having a shape corresponding to an outer shape of the medical supply in a central portion thereof.

The first sheet member 20 and the second sheet member 40 include abutting portions 22 and 42 surrounding an outer periphery of the storage recess 11 and facing each other, an annular seal portion 61 formed on the abutting portions 22 and 42 facing each other, surrounding the storage recess 11, and hermetically sealing the storage recess 11, and a non-seal portion 62 provided on an outer side of the annular seal portion 61.

At least one (in this example, the first sheet member 20) of the first sheet member 20 and the second sheet member 40 includes a linear or band-shaped breakable portion 23, the breakable portion 23 includes an opening start portion 24 including a start end on the non-seal portion 62, a peelable portion 25 continuous from the opening start portion 24 and crossing a part of the annular seal portion 61, and a breakable portion main part 26 continuous from the peelable portion 25 and crossing the storage recess 11, and the breakable portion 23 does not inhibit airtightness of the annular seal portion 61.

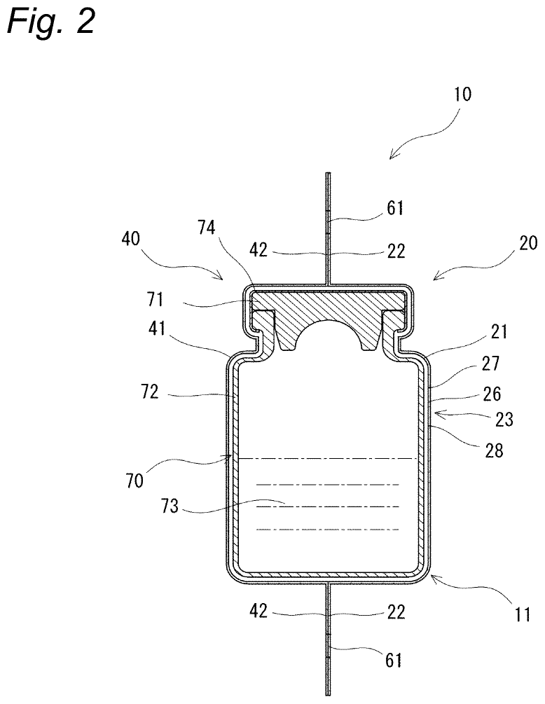

In the medical supply package 10 of this example, a general vial 70 is stored as the medical supply 70. As illustrated in FIG. 2, in the vial 70, a drug 73 is stored in a container 72 made of glass sealed with a rubber stopper 71. A peripheral edge of the rubber stopper 71 and a peripheral edge of an opening of the container 72 are covered with a covering member 74. The covering member 74 is preferably formed of aluminum, a heat-shrinkable film or the like, and is preferably in close contact with the rubber stopper 71 and the container 72. Note that, as the medical supply of the medical supply package, various medical supplies such as an ampoule, a syringe (including a prefilled syringe), an automatic drug solution administration device (for example, an insulin pump), and various medical devices can be employed in addition to the illustrated vial.

In this example, the first sheet member 20 and the second sheet member 40 are separate members, and the first sheet member 20 and the second sheet member 40 include the storage recesses 21 and 41 (first storage recess 21 and second storage recess 41), respectively, each having a shape corresponding to the outer shape of the medical supply 70 in the central portion thereof. Specifically, as illustrated in FIG. 2, the first sheet member 20 includes the first sheet-side recess 21 (first storage recess 21) having a shape corresponding to an outer shape of a substantial half portion of the medical supply 70, the second sheet member 40 includes the second sheet-side recess 41 (second storage recess 41) having a shape corresponding to an outer shape of a substantial half portion of the medical supply 70, and the first sheet-side recess 21 and the second sheet-side recess 41 form the storage recess 11 having a shape corresponding to an entire outer shape of the medical supply 70.

The first sheet member 20 and the second sheet member 40 are preferably made of resin having a certain degree of strength and hardness, and preferably have a certain degree of transparency. Examples of materials for forming the first sheet member 20 and the second sheet member 40 include polyolefin resins such as polypropylene and polyethylene, polyester resins such as polyethylene terephthalate, polystyrene resins, vinyl chloride and the like. Note that, as a material for forming the first sheet member and the second sheet member, a material having a multilayered structure in which various functional layers (oxygen impermeable layer, adhesive layer, printed layer and the like) are stacked on a base material layer formed of the above-described material may be used.

The first sheet member 20 and the second sheet member 40 include the abutting portions 22 and 42, respectively, the portions surrounding the outer periphery of the storage recess 11 (21 and 41) and facing each other. Specifically, as illustrated in FIGS. 1 and 2, the abutting portions 22 and 42 are planar portions extending outward from outer peripheral edges of the storage recesses 21 and 41, respectively. The outer shape (outer edge shape) of the abutting portions 22 and 42 is a rounded rectangular shape. Note that, the outer shape (outer edge shape) of the abutting portion is not limited to the illustrated shape, and is appropriately set. As illustrated in FIG. 2, the abutting portion 22 of the first sheet member 20 and the abutting portion 42 of the second sheet member 40 face each other to abut each other over an entire surface in a state in which the medical supply 70 is stored.

The annular seal portion 61 surrounding the storage recess 11 and hermetically sealing the storage recess 11 is formed on the abutting portions 22 and 42. Specifically, as illustrated in FIG. 1, the annular seal portion 61 having a substantially rectangular annular shape having a constant width is formed on the abutting portions 22 and 42 facing each other and abutting each other (in this example, the central portion of the abutting portions 22 and 42) so as to surround the storage recess 11 (refer to a cross-hatched portion in FIG. 1). The annular seal portion 61 is formed in such a manner that an outer edge is located on an inner side of the outer peripheral edge of the abutting portions 22 and 42 and an inner edge is located on an outer side of the outer peripheral edge of the storage recess 11. The annular seal portion 61 can be formed by adhering, fusing, welding or the like the first sheet member 20 (abutting portion 22) and the second sheet member 40 (abutting portion 42). The first sheet member 20 and the second sheet member 40 are fixed to each other in the annular seal portion 61.

The non-seal portion 62 is provided on the outer side of the annular seal portion 61. Specifically, in this example, as described above, the outer edge of the annular seal portion 61 is formed to be located on the inner side of the outer peripheral edge of the abutting portions 22 and 42, so that the first sheet member 20 and the second sheet member 40 are not fixed to (sealed) each other in a portion (a substantially rectangular annular portion having a constant width) between the outer edge of the annular seal portion 61 and the outer peripheral edge of the abutting portions 22 and 42. Herein, as illustrated in FIG. 1, the outer side on a left side of the annular seal portion 61 serves as the non-seal portion 62. An inner side of the annular seal portion 61 also serves as a non-seal portion (unsealed portion).

In this example, the first sheet member 20 includes the band-shaped breakable portion 23. The breakable portion 23 of the first sheet member 20 includes a first breakable linear portion 27 extending over a substantially entire length of the breakable portion 23, and a second breakable linear portion 28 facing (in this example, substantially parallel to) the first breakable linear portion 27 and extending over the substantially entire length of the breakable portion 23. In other words, the band-shaped breakable portion 23 is formed on the first sheet member 20 by the first breakable linear portion 27 and the second breakable linear portion 28. As will be described later in detail, the first breakable linear portion 27 and the second breakable linear portion 28 are cuts (fragile portions) intermittently formed on the first sheet member 20, and have lower strength (are broken more easily) than other portions of the first sheet member 20 (portions where the first breakable linear portion 27 and the second breakable linear portion 28 are not formed).

Note that, a width of the band-shaped breakable portion 23 is preferably 1 to 20 mm, more preferably 2 to 15 mm, and especially preferably 3 to 10 mm. The breakable portion 23 may have a width but a narrow width (specifically, 2 mm or less), and may be substantially linear.

The breakable portion 23 includes the opening start portion 24 including the start end on the non-seal portion 62. In this example, as illustrated in FIG. 1, one end portion (left end portion in FIG. 1) of the breakable portion 23 serves as the opening start portion 24. Because the opening start portion 24 is a portion having a constant width interposed between the first breakable linear portion 27 and the second breakable linear portion 28, and the first sheet member 20 is not fixed to the second sheet member 40 in the opening start portion 24, it is easy to grip the opening start portion 24. As a result, it is easy to start breaking the breakable portion 23 (opening the medical supply package 10). Note that, in the opening start portion 24, two small notches 29 and 29 having a substantially V shape are formed on upper and lower sides on an outer end thereof, so that the opening start portion 24 can be more easily gripped and it is easy to start breaking the breakable portion 23 (opening the medical supply package 10).

The breakable portion 23 includes the peelable portion 25 continuous from the opening start portion 24 and crossing a part of the annular seal portion 61. In this example, as illustrated in FIG. 1, in the band-shaped breakable portion 23, a portion corresponding to a left side portion of the annular seal portion 61 having a substantially rectangular annular shape serves as the peelable portion 25. The peelable portion 25 crosses the left side portion of the annular seal portion 61 having a substantially rectangular annular shape from left to right. Although the first sheet member 20 is fixed to the second sheet member 40 in the peelable portion 25, when breakage of the breakable portion 23 is started from the opening start portion 24, the peelable portion 25 continuous from the opening start portion 24 is easily peeled from the second sheet member 40.

The breakable portion 23 includes the breakable portion main part (breakable portion central part) 26 continuous from the peelable portion 25 and crossing the storage recess 11 (first storage recess 21). Specifically, as illustrated in FIG. 1, the breakable portion main part 26 extends continuously from the peelable portion 25 (from a portion on a side opposite to the opening start portion 24 side of the peelable portion 25), and crosses the non-seal portion and the storage recess 11 in the annular seal portion 61.

In this example, the medical supply package 10 includes an opposing non-seal portion 63 provided in a position opposed to the non-seal portion 62 in a width direction (right-to-left direction in FIG. 1) on the outer side of the annular seal portion 61, and the breakable portion 23 includes a second peelable portion 30 continuous from the breakable portion main part 26 and crossing a part of the annular seal portion 61, and a second opening start portion 31 continuous from the second peelable portion 30 and including a start end in the opposing non-seal portion 63. Therefore, an opening operation can be performed from both the first opening start portion 24 side and the second opening start portion 31 side, and the opening operation can be performed regardless of a direction at the time of opening operation of the medical supply package 10 and a dominant hand of an opening person.

Specifically, as illustrated in FIG. 1, in the band-shaped breakable portion 23, a portion continuous from the breakable portion main part 26 and corresponding to a right side portion of the annular seal portion 61 having a substantially rectangular annular shape serves as the second peelable portion 30. The second peelable portion 30 crosses the right side portion of the annular seal portion 61 having a substantially rectangular annular shape from left to right. Although the first sheet member 20 is fixed to the second sheet member 40 in the second peelable portion 30, when breakage of the second peelable portion 30 is progressed from the breakable portion main part 26, the second peelable portion 30 is easily peeled from the second sheet member 40.

As described above, in the medical supply package 10, the first sheet member 20 and the second sheet member 40 are not fixed to (sealed) each other in a portion (substantially rectangular annular portion having a constant width) between the outer edge of the annular seal portion 61 and the outer peripheral edge of the abutting portions 22 and 42. As a result, as illustrated in FIG. 1, the opposing non-seal portion 63 is provided on the outer side on a right side of the annular seal portion 61. The breakable portion 23 includes the second opening start portion 31 continuous from the second peelable portion 30 and including a start end (terminal end) in the opposing non-seal portion 63. In this example, as illustrated in FIG. 1, the other end portion (right end portion in FIG. 1) of the breakable portion 23 serves as the second opening start portion 31. Because the second opening start portion 31 is a portion having a constant width interposed between the first breakable linear portion 27 and the second breakable linear portion 28, and the first sheet member 20 is not fixed to the second sheet member 40 in the second opening start portion 31, it is possible to start breaking the breakable portion 23 from the second opening start portion 31, and it is easy to complete the breakage of the breakable portion 23 broken from the opening start portion 24.

In the medical supply package 10 of this example, the breakable portion 23 is provided over the entire length in the width direction (right-to-left direction in FIG. 1) of the medical supply package 10 (first sheet member 20). Specifically, the first breakable linear portion 27 and the second breakable linear portion 28 are provided over the entire length in the width direction of the first sheet member 20, and the opening start portion 24, the peelable portion 25, the breakable portion main part 26, the second peelable portion 30, and the second opening start portion 31 are provided in this order from a left side end to a right side end of the band-shaped breakable portion 23. Because the first breakable linear portion 27 and the second breakable linear portion 28 are provided over the entire length in the width direction of the first sheet member 20, the breakable portion 23 can be cut off from the medical supply package 10. As a result, when the medical supply package 10 is opened and the medical supply 70 is taken out, it is possible to open wide the medical supply package 10 while gripping the portion from which the breakable portion 23 is cut off (the portion in which the breakable portion 23 has been formed.

The breakable portion 23 does not inhibit airtightness of the annular seal portion 61. In this example, the first breakable linear portion 27 and the second breakable linear portion 28 are formed of intermittently formed cuts, and do not penetrate the first sheet member 20 or the second sheet member 40 provided with the breakable portion 23 in the annular seal portion 61 and/or on the inner side of the annular seal portion 61.

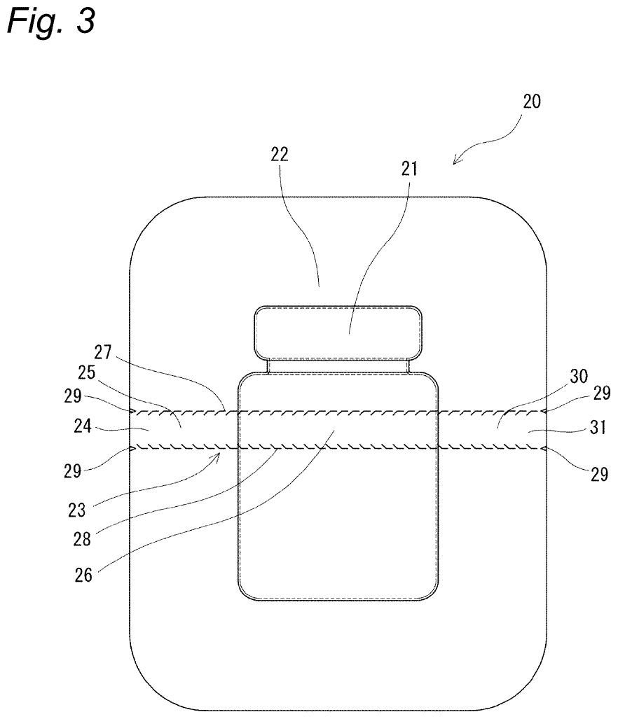

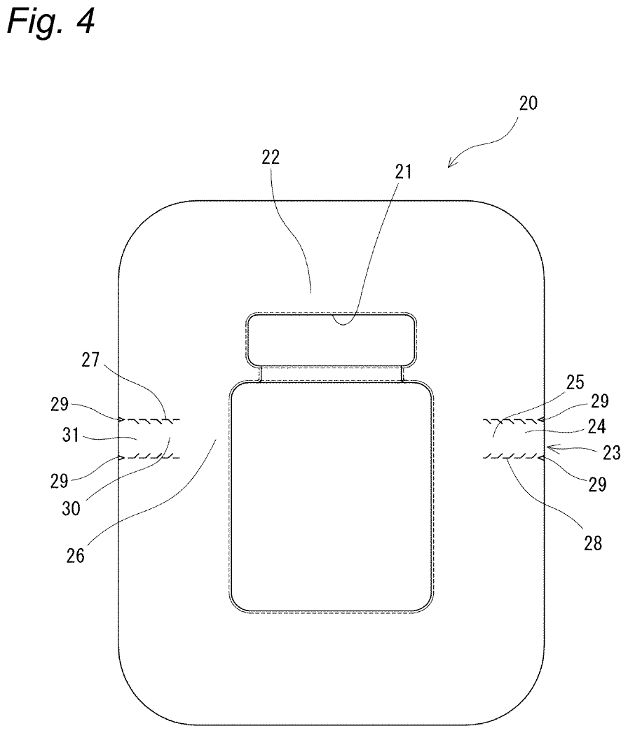

In this example, as illustrated in FIGS. 3 and 4, at a formation site of the opening start portion 24, the peelable portion 25, the second peelable portion 30, and the second opening start portion 31, cuts forming the first breakable linear portion 27 and the second breakable linear portion 28 penetrate the first sheet member 20. However, in the breakable portion main part 26 (an inner side of the annular seal portion 61), the cuts forming the first breakable linear portion 27 and the second breakable linear portion 28 are provided only on an outer layer side of the first sheet member 20, and do not penetrate the first sheet member 20 as illustrated in FIG. 4. Note that, in FIGS. 3 and 4, assuming that the cuts forming the first breakable linear portion 27 and the second breakable linear portion 28 are formed on the first sheet member 20 from a front surface (surface on an outer side of the medical supply package 10 and is illustrated in FIG. 3), a portion in which the first sheet member 20 is not penetrated is not exposed to a back surface (surface on an inner side of the medical supply package 10 and is illustrated in FIG. 4).

In other words, the cuts forming the first breakable linear portion 27 and the second breakable linear portion 28 in the breakable portion main part 26 extend from the outer surface toward the inner surface of the first sheet member 20, terminate in a wall of the first sheet member 20, and do not reach the inner surface.

As a result, while securing airtightness of the medical supply package 10, especially, in the portion in which the cuts forming the first breakable linear portion 27 and the second breakable linear portion 28 penetrate the first sheet member 20 (opening start portion 24, peelable portion 25, second peelable portion 30, and second opening start portion 31), the breakable portion 23 is easily broken (and peeled).

Note that, the first breakable linear portion 27 and the second breakable linear portion 28 in the breakable portion main part 26 may be that not exposed on the front surface of the first sheet member 20, specifically, may include an outer layer having no cuts as long as they do not inhibit breakage. In this case, the first breakable linear portion 27 and the second breakable linear portion 28 are formed in the wall of the first sheet member 20.

Note that, in the medical supply package, the cuts forming the first breakable linear portion and the second breakable linear portion may be that not penetrating the first sheet member and/or the second sheet member even in the annular seal portion (the peelable portion and the second peelable portion), and may that not penetrating the first sheet member and/or the second sheet member over the entire length.

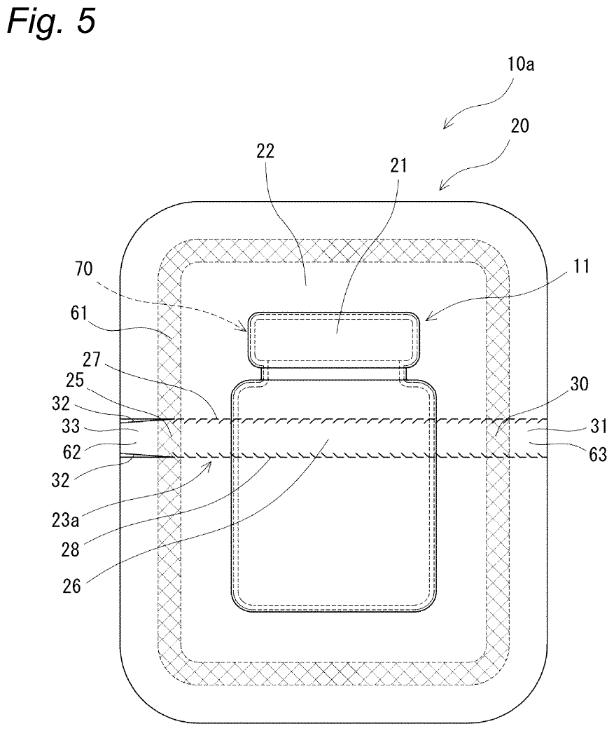

As in a medical supply package 10a illustrated in FIG. 5, an opening start portion of a breakable portion 23a may be a tab (opening start portion) 33 formed of two slits 32 and 32 provided on upper and lower sides that can be gripped. One end of the two slits 32 and 32 may be located at an outer edge of the non-seal portion 62 of the first sheet member 20 or the second sheet member 40 provided with the breakable portion 23a, and the other end may be located near or on an inner side of the outer edge of the peelable portion 25.

Specifically, as illustrated in FIG. 5, in the medical supply package 10a, the two slits 32 and 32 having a substantially V shape extending in an extending direction (right-to-left direction in FIG. 5) of the breakable portion 23a are formed on the upper and lower sides of the opening start portion 33, and a portion interposed between the slits 32 and 32 serves as the tab (opening start portion) 33 having a substantially trapezoidal shape. As a result, the opening start portion (tab 33) is easily gripped, and breakage of the breakable portion 23a (opening of the medical supply package 10a) from the opening start portion (tab 33) is easily started.

In the medical supply package 10a, one end of the two slits 32 and 32 is located at the outer edge of the non-seal portion 62 of the first sheet member 20 provided with the breakable portion 23a, and the other end is located on the inner side of the outer edge of the peelable portion 25. This makes it easier to peel the peelable portion 25 continuous from the opening start portion (tab 33).

Note that, also in the second opening start portion 31 in FIG. 5, two slits may be provided on upper and lower sides to form a tab that can be gripped. As a result, the second opening start portion (tab) is easily gripped, breakage of the breakable portion (opening of the medical supply package) can be started from the second opening start portion (tab), and the breakage of the breakable portion broken from the opening start portion can be easily completed.

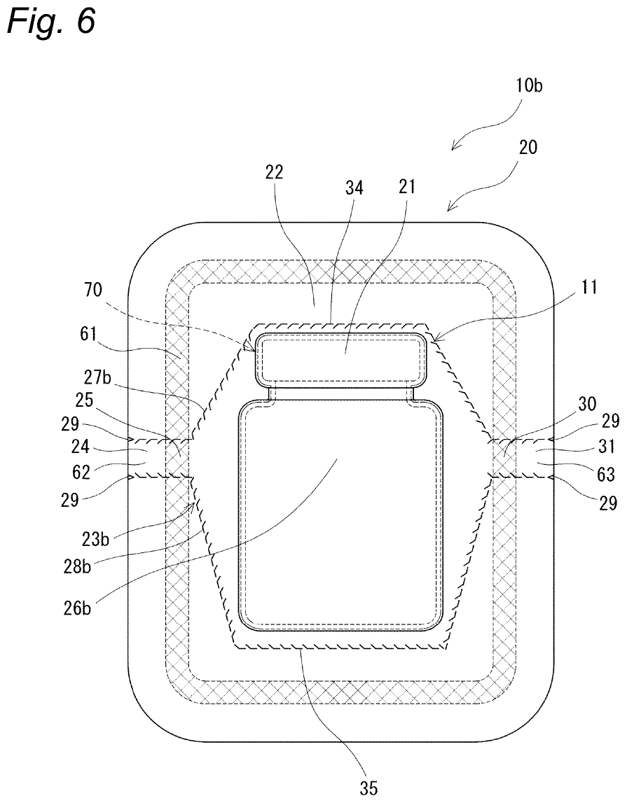

As in a medical supply package 10b illustrated in FIG. 6, a first breakable linear portion 27b may include an upper portion surrounding portion 34 surrounding an upper portion side of the storage recess 11, and a second breakable linear portion 28b may include a lower portion surrounding portion 35 surrounding a lower portion side of the storage recess 11. Specifically, as illustrated in FIG. 6, on the inner side of the annular seal portion 61 (a breakable portion main part 26b of a breakable portion 23b), the first breakable linear portion 27b is formed so as to bypass the storage recess 11 (first storage recess 21) on an upper side. A second breakable linear portion 28b is formed so as to bypass the storage recess 11 (first storage recess 21) on a lower side. Herein, the first breakable linear portion 27b and the second breakable linear portion 28b are not formed at a formation site of the storage recess 11 (first storage recess 21), and the breakable portion main part 26b is formed so as to cross (include) an entire storage recess 11 (first storage recess 21). As a result, it becomes easy to take out the medical supply 70 after opening the medical supply package 10b.

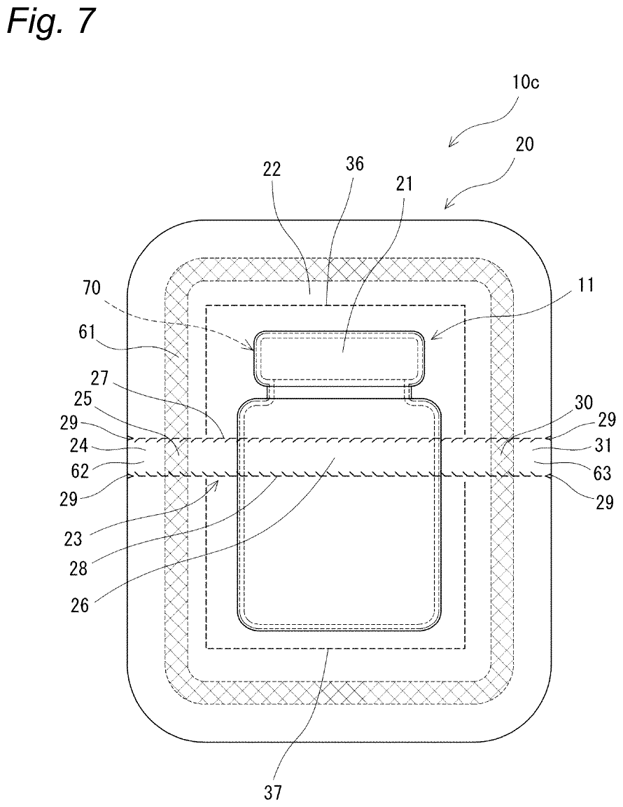

As illustrated in FIG. 7, a medical supply package 10c may include an upper portion expanding breakable linear portion 36 located above the first breakable linear portion 27 of the breakable portion 23 and surrounding the upper portion side of the storage recess 11, and a lower portion expanding breakable linear portion 37 located below the second breakable linear portion 28 of the breakable portion 23 and surrounding the lower portion side of the storage recess 11. Specifically, as illustrated in FIG. 7, on the inner side of the annular seal portion 61, the upper portion expanding breakable linear portion 36 in a substantially U shape opening downward is formed above the first breakable linear portion 27 so as to surround the upper portion side of the storage recess 11 (first storage recess 21). The lower portion expanding breakable linear portion 37 in a substantially U shape opening upward is formed below the second breakable linear portion 28 so as to surround the lower portion side of the storage recess 11 (first storage recess 21). A lower end of the upper portion expanding breakable linear portion 36 is adjacent to the first breakable linear portion 27, and an upper end of the lower portion expanding breakable linear portion 37 is adjacent to the second breakable linear portion 28. As a result, after the breakable portion 23 is cut off, the inner side portion of the annular seal portion 61 is broken along the upper portion expanding breakable linear portion 36 and the lower portion expanding breakable linear portion 37 (the inner side portion of the upper portion expanding breakable linear portion 36 and the lower portion expanding breakable linear portion 37 is cut off) from this cut-off portion (portion in which the first breakable linear portion 27 and the second breakable linear portion 28 are formed), so that the stored medical supply 70 can be easily taken out.

Note that, the upper portion expanding breakable linear portion 36 and the lower portion expanding breakable linear portion 37 can be formed by cuts formed intermittently or the like, and because they are formed on the inner side of the annular seal portion 61, it is preferable that these cuts do not penetrate the first sheet member 20 or the second sheet member 40 (herein, the first sheet member 20) provided with the breakable portion 23.

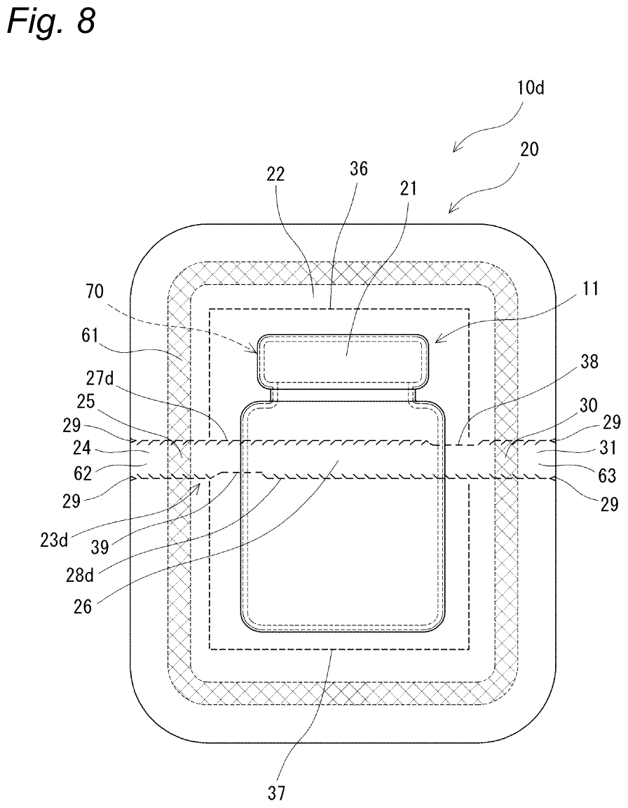

As illustrated in FIG. 8, as the medical supply package of the present invention, a storage recess upper portion expanded time grip portion forming portion 38 and a storage recess lower portion expanded time grip portion forming portion 39 may be provided in the inner side portion of the annular seal portion 61. Specifically, as illustrated in FIG. 8, by deforming (herein, bypassing slightly downward) a part of a first breakable linear portion 27d, it is possible to form the storage recess upper portion expanded time grip portion forming portion 38 slightly protruding downward. By deforming (herein, bypassing slightly upward) a part of a second breakable linear portion 28d, it is possible to form the storage recess lower portion expanded time grip portion forming portion 39 slightly protruding upward. As a result, after a breakable portion 23d is cut off, a storage recess upper portion expanded time grip portion and a storage recess lower portion expanded time grip portion can be formed at a formation site of the storage recess upper portion expanded time grip portion forming portion 38 and the storage recess lower portion expanded time grip portion forming portion 39, and a medical supply package 10d can be easily opened by gripping the grip portions.

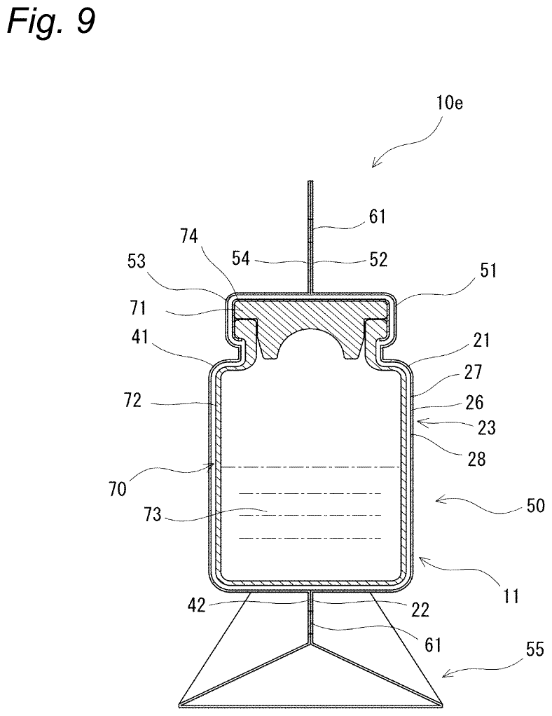

Note that, in the above-described example, the first sheet member and the second sheet member are separate members, but the first sheet member and the second sheet member may be integrated. In this case, for example, as in a medical supply package 10e illustrated in FIG. 9, by folding an integrated sheet member 50, an abutting portion 52 of a first sheet portion 51 and an abutting portion 54 of a second sheet portion 53 may face each other to form the annular seal portion 61. As illustrated in FIG. 9, a pedestal portion 55 that enables the medical supply package 10e to stand by itself may be provided in a folded portion of the integrated sheet member 50.

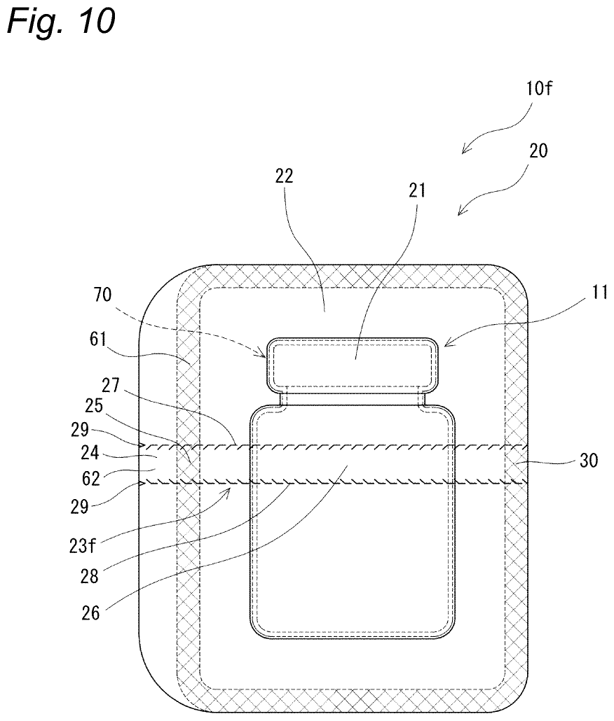

It is not necessary to provide the non-seal portion over an entire circumference of the medical supply package, and for example, as in a medical supply package 10f illustrated in FIG. 10, it is possible to provide the non-seal portion 62 only in a portion in which the opening start portion 24 is provided (only in a left side portion in FIG. 10). In other words, the medical supply package 10f does not include the opposing non-seal portion provided in a position opposed to the non-seal portion 62 on the outer side of the annular seal portion 61, and the breakable portion 23f of the medical supply package 10f does not include the second opening start portion.

Note that, as the medical supply package of the present invention, the storage recess having the shape corresponding to the entire outer shape of the medical supply may be provided on the first sheet member or the second sheet member (only one of the first sheet member and the second sheet member). In this case, the other of the first sheet member and the second sheet member may be a substantially planar member without the storage recess provided, or a relatively thin film-shaped member capable of closing the storage recess.

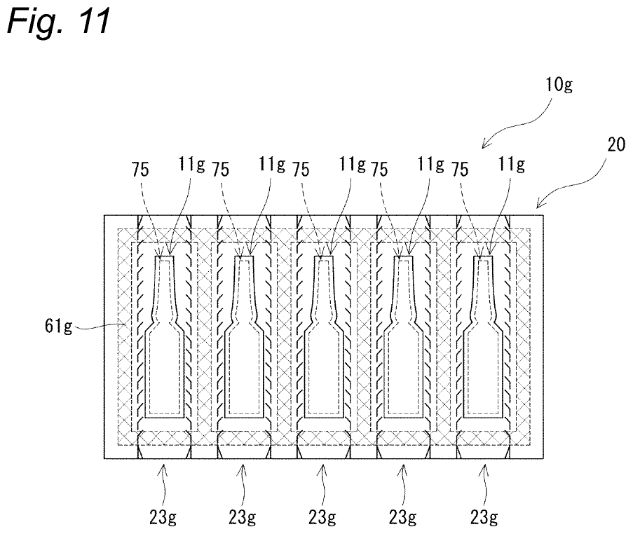

In the medical supply package of the present invention, as in a medical supply package 10g illustrated in FIG. 11, a plurality of storage recesses 11g may be provided, and breakable portions 23g corresponding to the respective storage recesses 11g may be provided. In a medical supply package 10g, a plurality of (herein, five) ampoules 75 is stored as medical supplies, and a plurality of (herein, five) storage recesses 11g having a shape corresponding to an outer shape of the ampoule 75 is provided. The medical supply package 10g includes an annular seal portion 61g surrounding each storage recess 11g and hermetically sealing the storage recess 11g. Note that, the annular seal portion 61g has a shape in which a plurality of annular seal portions is integrated. A band-shaped breakable portion 23g is provided for each storage recess 11g. As a result, airtightness in each storage recess 11g is secured, and the medical supplies (ampoules 75) can be easily taken out one by one.

Note that, in the above-described example, the breakable portion is provided only on the first sheet member, but the breakable portion may be provided on the second sheet member.

Note that, in the above-described example, the breakable linear portion (first breakable linear portion and second breakable linear portion) of the breakable portion is formed by cuts formed intermittently, but there is no limitation. The breakable linear portion of the breakable portion may be formed by, for example, perforations, a fold, intermittently formed dots, a continuously or intermittently formed thin portion (half cut portion) or the like.

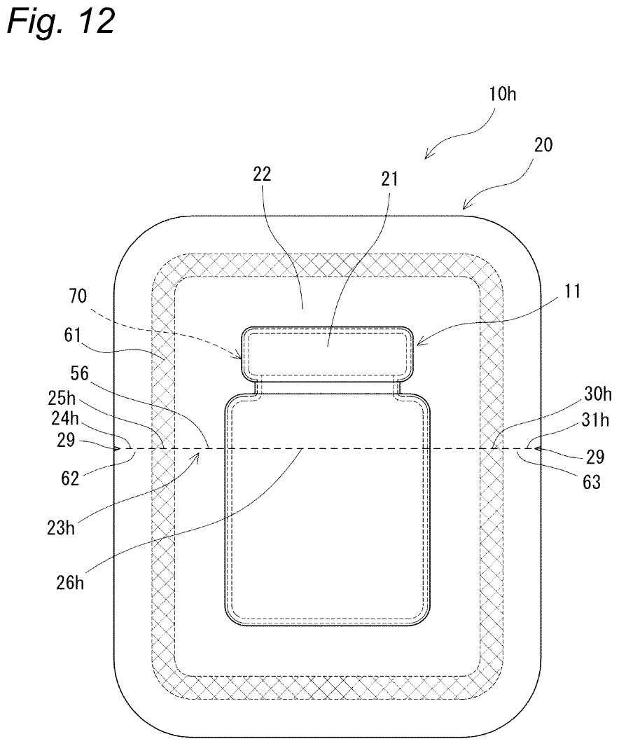

As in a medical supply package 10h illustrated in FIG. 12, the breakable portion may be a linear breakable portion 23h. The breakable portion 23h includes one breakable linear portion 56 formed by perforations. The breakable portion 23h includes, in order from the left side in FIG. 12, an opening start portion 24h, a peelable portion 25h, a breakable portion main part 26h, a second peelable portion 30h, and a second opening start portion 31h.

Then, another embodiment of the medical supply package of the present invention will be described with reference to an example illustrated in the drawings. Note that, in the following description, components substantially similar to those of the medical supply package 10 described above are denoted by corresponding names and reference signs, and a detailed description thereof will be omitted.

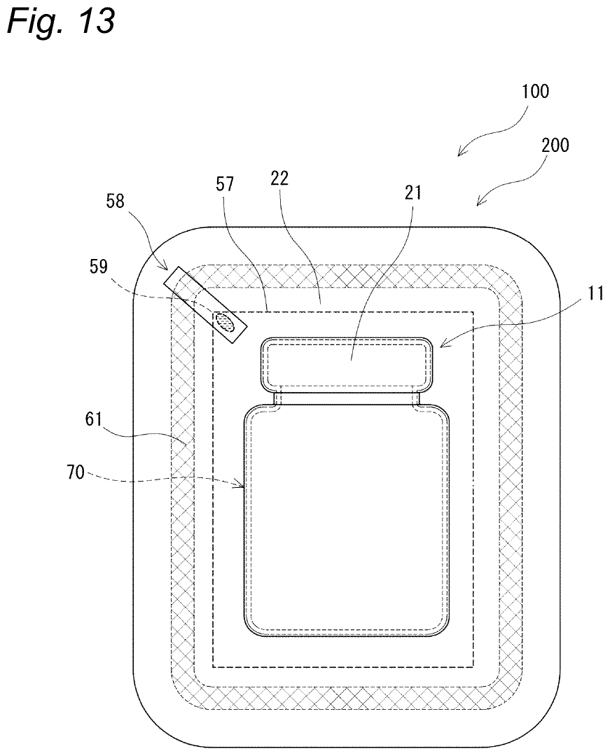

As illustrated in FIG. 13, a medical supply package 100 includes a first sheet member 200 and a second sheet member 40 facing the first sheet member 200, and stores a medical supply 70 between the first sheet member 200 and the second sheet member 40. The first sheet member 200 and/or the second sheet member 40 (herein, the first sheet member 200 and the second sheet member 40) includes a storage recess 11 (first storage recess 21 and second storage recess 42) having a shape corresponding to an outer shape of the medical supply 70 in a central portion thereof. The first sheet member 200 and the second sheet member 40 include abutting portions 22 and 42 surrounding an outer periphery of the storage recess 11 and facing each other, and an annular seal portion 61 formed on the abutting portions 22 and 42 facing each other, surrounding the storage recess 11, and hermetically sealing the storage recess 11. At least one (herein, the first sheet member 200) of the first sheet member 200 and the second sheet member 40 includes a breakable linear portion 57 surrounding the storage recess 11 and located on an inner side of the annular seal portion 61. The medical supply package 100 includes an opening tab 58 one end of which is fixed to an inner edge of the breakable linear portion 57 and the other end of which is a free end, in which the breakable linear portion 57 is formed of intermittently formed cuts, and do not penetrate the first sheet member 200 or the second sheet member 40 (herein, the first sheet member 200) provided with the breakable linear portion 57.

Specifically, as illustrated in FIG. 13, the breakable linear portion 57 is formed on the first sheet member 200 of the medical supply package 100 so as to surround the storage recess 11 in the inner side portion of the annular seal portion 61. In other words, the breakable linear portion 57 is formed in a portion on an outer side of the storage recess 11 (first storage recess 21) and an inner side of the annular seal portion 61 in the abutting portion 22 of the first sheet member 200. Herein, the breakable linear portion 57 is an annular (substantially rectangular annular) breakable linear portion. The breakable linear portion 57 is formed by cuts formed intermittently and does not penetrate the first sheet member 200. As a result, the breakable linear portion 57 does not inhibit airtightness of the annular seal portion 61.

In the medical supply package 100, one end of the opening tab 58 is fixed to the inner edge (herein, an upper left inner edge in FIG. 13) of the breakable linear portion 57. In the present example, the breakable linear portion 57 includes a corner 59 having an edge (corner without an R shape formed), and one end of the opening tab 58 is fixed to a position on an inner side of the corner 59, the position near the same. More specifically, in the present example, as illustrated in FIG. 13, an upper left corner 59 of the breakable linear portion 57 is a corner having an edge (herein, a corner at a substantially right angle in which an R shape is not formed), and one end of the opening tab 58 is fixed to a position on an inner side of the corner 59, the position near the same. As a result, when the other end (free end) of the opening tab 58 is gripped and pulled, a stress concentrates on the corner 59 of the breakable linear portion 57 via one end of the fixed opening tab 58, and the breakable linear portion 57 is easily broken (the medical supply package 100 is easily opened) using the opening tab 58.

In the present example, the breakable linear portion 57 is an annular breakable linear portion, and a portion surrounded by the annular breakable linear portion 57 can be cut off from the medical supply package 100. As a result, it becomes easier to take out the medical supply 70 from the medical supply package 100.

A medical supply package of the present invention includes a first sheet member and a second sheet member facing the first sheet member, and stores a medical supply between the first sheet member and the second sheet member, in which the first sheet member and/or the second sheet member includes a storage recess having a shape corresponding to an outer shape of the medical supply in a central portion, the first sheet member and the second sheet member includes abutting portions surrounding an outer periphery of the storage recess and facing each other, an annular seal portion formed on the abutting portions facing each other, surrounding the storage recess, and hermetically sealing the storage recess, and a non-seal portion provided on an outer side of the annular seal portion, and at least one of the first sheet member and the second sheet member includes a linear or band-shaped breakable portion, the breakable portion includes an opening start portion including a start end in the non-seal portion, a peelable portion continuous from the opening start portion and crossing a part of the annular seal portion, and a breakable portion main part continuous from the peelable portion and crossing the storage recess, and the breakable portion does not inhibit airtightness of the annular seal portion. As a result, in the medical supply package, the stored medical supply can be easily taken out while securing airtightness.

A medical supply package of the present invention includes a first sheet member and a second sheet member facing the first sheet member, and stores a medical supply between the first sheet member and the second sheet member, in which the first sheet member and/or the second sheet member includes a storage recess having a shape corresponding to an outer shape of the medical supply in a central portion, the first sheet member and the second sheet member includes abutting portions surrounding an outer periphery of the storage recess and facing each other, and an annular seal portion formed on the abutting portions facing each other, surrounding the storage recess, and hermetically sealing the storage recess, at least one of the first sheet member and the second sheet member includes a breakable linear portion surrounding an inner side of the annular seal portion and the storage recess, and the medical supply package includes an opening tab one end of which is fixed to an inner edge of the breakable linear portion and the other end of which is a free end, in which the breakable linear portion is formed of intermittently formed cuts, and do not penetrate the first sheet member or the second sheet member provided with the breakable linear portion. As a result, in the medical supply package, the airtightness.

The medical supply package of the present invention is as follows.
(1) A medical supply package storing a medical supply,
the medical supply package including a first sheet member and a second sheet member facing the first sheet member, the medical supply package storing the medical supply between the first sheet member and the second sheet member, in which
the first sheet member and/or the second sheet member includes a storage recess having a shape corresponding to an outer shape of the medical supply in a central portion,
the first sheet member and the second sheet member includes abutting portions surrounding an outer periphery of the storage recess and facing each other, an annular seal portion formed on the abutting portions facing each other, surrounding the storage recess, and hermetically sealing the storage recess, and a non-seal portion provided on an outer side of the annular seal portion, and
at least one of the first sheet member and the second sheet member includes a linear or band-shaped breakable portion, the breakable portion includes an opening start portion including a start end in the non-seal portion, a peelable portion continuous from the opening start portion and crossing a part of the annular seal portion, and a breakable portion main part continuous from the peelable portion and crossing the storage recess, and the breakable portion does not inhibit airtightness of the annular seal portion.

The medical supply package includes a first sheet member and a second sheet member facing the first sheet member, and stores a medical supply between the first sheet member and the second sheet member, in which the first sheet member and/or the second sheet member includes a storage recess having a shape corresponding to an outer shape of the medical supply in a central portion, the first sheet member and the second sheet member includes abutting portions surrounding an outer periphery of the storage recess and facing each other, an annular seal portion formed on the abutting portions facing each other, surrounding the storage recess, and hermetically sealing the storage recess, and a non-seal portion provided on an outer side of the annular seal portion, and at least one of the first sheet member and the second sheet member includes a linear or band-shaped breakable portion, the breakable portion includes an opening start portion including a start end in the non-seal portion, a peelable portion continuous from the opening start portion and crossing a part of the annular seal portion, and a breakable portion main part continuous from the peelable portion and crossing the storage recess, and the breakable portion does not inhibit airtightness of the annular seal portion. As a result, in the medical supply package, the airtightness.

The above-described embodiment may be configured as follows.
(2) The medical supply package according to (1) above, in which the first sheet member includes a first sheet-side recess, the second sheet member includes a second sheet-side recess, and the storage recess is formed by the first sheet-side recess and the second sheet-side recess.
(3) The medical supply package according to (1) or (2) above, including an opposing non-seal portion provided in a position opposed to the non-seal portion on an outer side of the annular seal portion, in which the breakable portion includes a second peelable portion continuous from the breakable portion main part and crossing a part of the annular seal portion, and a second opening start portion continuous from the second peelable portion and including a start end in the opposing non-seal portion.
(4) The medical supply package according to any one of (1) to (3) above, in which the breakable portion is able to be cut off from the medical supply package.
(5) The medical supply package according to any one of (1) to (4) above, in which the breakable portion includes a first breakable linear portion extending over a substantially entire length of the breakable portion, and a second breakable linear portion facing the first breakable linear portion and extending over the substantially entire length of the breakable portion.
(6) The medical supply package according to (5) above, in which the first breakable linear portion and the second breakable linear portion are formed of intermittently formed cuts, and do not penetrate the first sheet member or the second sheet member provided with the breakable portion in the annular seal portion and/or on an inner side of the annular seal portion.
(7) The medical supply package according to (5) or (6) above, in which the first breakable linear portion includes an upper portion surrounding portion surrounding an upper portion side of the storage recess, and the second breakable linear portion includes a lower portion surrounding portion surrounding a lower portion side of the storage recess.
(8) The medical supply package according to (5) or (6) above, in which the medical supply package includes an upper portion expanding breakable linear portion located above the first breakable linear portion of the breakable portion and surrounding an upper portion side of the storage recess, and a lower portion expanding breakable linear portion located below the second breakable linear portion of the breakable portion and surrounding a lower portion side of the storage recess.
(9) The medical supply package according to any one of (1) to (8) above, in which the opening start portion of the breakable portion is a tab formed of two slits provided on upper and lower sides that can be gripped.
(10) The medical supply package according to (9) above, in which one end of the two slits is located at an outer edge of the non-seal portion of the first sheet member or the second sheet member provided with the breakable portion, and the other end is located near or on an inner side of the outer edge of the peelable portion.

The medical supply package of the present invention is as follows.
(11) A medical supply package storing a medical supply, the medical supply package including
a first sheet member and a second sheet member facing the first sheet member,
the medical supply package storing the medical supply between the first sheet member and the second sheet member, in which
the first sheet member and/or the second sheet member includes a storage recess having a shape corresponding to an outer shape of the medical supply in a central portion,
the first sheet member and the second sheet member includes abutting portions surrounding an outer periphery of the storage recess and facing each other, and an annular seal portion formed on the abutting portions facing each other, surrounding the storage recess, and hermetically sealing the storage recess,
at least one of the first sheet member and the second sheet member includes a breakable linear portion surrounding an inner side of the annular seal portion and the storage recess, and
the medical supply package includes an opening tab one end of which is fixed to an inner edge of the breakable linear portion and the other end of which is a free end, in which the breakable linear portion is formed of intermittently formed cuts, and do not penetrate the first sheet member or the second sheet member provided with the breakable linear portion.

The medical supply package includes a first sheet member and a second sheet member facing the first sheet member, and stores a medical supply between the first sheet member and the second sheet member, in which the first sheet member and/or the second sheet member includes a storage recess having a shape corresponding to an outer shape of the medical supply in a central portion, the first sheet member and the second sheet member includes abutting portions surrounding an outer periphery of the storage recess and facing each other, and an annular seal portion formed on the abutting portions facing each other, surrounding the storage recess, and hermetically sealing the storage recess, at least one of the first sheet member and the second sheet member includes a breakable linear portion surrounding an inner side of the annular seal portion and the storage recess, and the medical supply package includes an opening tab one end of which is fixed to an inner edge of the breakable linear portion and the other end of which is a free end, in which the breakable linear portion is formed of intermittently formed cuts, and do not penetrate the first sheet member or the second sheet member provided with the breakable linear portion. As a result, in the medical supply package, the stored medical supply can be easily taken out while securing airtightness.

The above-described embodiment may be configured as follows.
(12) The medical supply package according to (11) above, in which the breakable linear portion includes a corner having an edge, and the one end of the opening tab is fixed to a position on an inner side of the corner, the position near the corner.
(13) The medical supply package according to (11) or (12) above, in which the breakable linear portion is an annular breakable linear portion.
(14) The medical supply package according to any one of (11) to (13), in which the breakable linear portion is an annular breakable linear portion, and a portion surrounded by the annular breakable linear portion is able to be cut off from the medical supply package.

1. A medical supply package storing a medical supply, the medical supply package comprising:
a first sheet member; and
a second sheet member facing the first sheet member; wherein:
the medical supply is stored between the first sheet member and the second sheet member;
the first sheet member and/or the second sheet member comprises a storage recess having a shape corresponding to an outer shape of the medical supply in a central portion;
the first sheet member and the second sheet member comprise:
abutting portions surrounding an outer periphery of the storage recess and facing each other,
an annular seal portion formed on the abutting portions and surrounding and hermetically sealing the storage recess, and
a non-seal portion provided on an outer side of the annular seal portion; and
at least one of the first sheet member and the second sheet member comprises a linear or band-shaped breakable portion, the breakable portion comprising:
an opening start portion including a start end in the non-seal portion,
a peelable portion continuous from the opening start portion and crossing a part of the annular seal portion, and
a breakable portion main part continuous from the peelable portion and crossing the storage recess, wherein the breakable portion does not inhibit airtightness of the annular seal portion.

2. The medical supply package according to claim 1, wherein:
the first sheet member comprises a first sheet-side recess;
the second sheet member comprises a second sheet-side recess; and
the storage recess is formed by the first sheet-side recess and the second sheet-side recess.

3. The medical supply package according to claim 1, wherein:
the first sheet member and the second sheet member comprise an opposing non-seal portion provided in a position opposite the non-seal portion on an outer side of the annular seal portion; wherein:
the breakable portion comprises:
a second peelable portion continuous from the breakable portion main part and crossing a part of the annular seal portion, and
a second opening start portion continuous from the second peelable portion and including a start end in the opposing non-seal portion.

4. The medical supply package according to claim 1, wherein the breakable portion is able to be cut off from the medical supply package.

5. The medical supply package according to claim 1, wherein the breakable portion comprises a first breakable linear portion and a second breakable linear portion facing the first breakable linear portion, where the first and second breakable linear portions extends over substantially an entire length of the breakable portion.

6. The medical supply package according to claim 5, wherein:
the first breakable linear portion and the second breakable linear portion are formed of intermittently formed cuts;
the cuts of the first breakable linear portion and the second breakable linear portion do not penetrate the first sheet member and do not penetrate the second sheet member in the annular seal portion; and
the cuts of the first breakable linear portion and the second breakable linear portion do not penetrate the first sheet member and do not penetrate the second sheet member on an inner side of the annular seal portion.

7. The medical supply package according to claim 5, wherein:
the first breakable linear portion comprises an upper portion surrounding portion surrounding an upper portion side of the storage recess; and
the second breakable linear portion comprises a lower portion surrounding portion surrounding a lower portion side of the storage recess.

8. The medical supply package according to claim 5, comprising:
an upper portion expanding breakable linear portion located above the first breakable linear portion and surrounding an upper portion side of the storage recess; and
a lower portion expanding breakable linear portion located below the second breakable linear portion and surrounding a lower portion side of the storage recess.

9. The medical supply package according to claim 1, wherein the opening start portion of the breakable portion is a grippable tab formed by two slits provided on upper and lower sides.

10. The medical supply package according to claim 9, wherein one end of each slit is located at an outer edge of the non-seal portion of the first sheet member or the second sheet member provided with the breakable portion, and the other end of each slit located near or on an inner side of the outer edge of the peelable portion.

11. A medical supply package storing a medical supply, the medical supply package comprising:
a first sheet member; and
a second sheet member facing the first sheet member; wherein:
the medical supply is stored between the first sheet member and the second sheet member;
the first sheet member and/or the second sheet member comprises a storage recess having a shape corresponding to an outer shape of the medical supply in a central portion;
the first sheet member and the second sheet member comprise:
abutting portions surrounding an outer periphery of the storage recess and facing each other, and
an annular seal portion formed on the abutting portions, and surrounding and hermitically sealing the storage recess;
at least one of the first sheet member and the second sheet member includes a breakable linear portion surrounding the storage recess and located on an inner side of the annular seal portion; and
the medical supply package comprises an opening tab one end of which is fixed to an inner edge of the breakable linear portion and the other end of which is a free end;
the breakable linear portion is formed of intermittently formed cuts that do not penetrate the first sheet member and do not penetrate the second sheet member.

12. The medical supply package according to claim 11, wherein the breakable linear portion comprises a corner having an edge, and the one end of the opening tab is fixed to a position near the corner on an inner side of the corner.

13. The medical supply package according to claim 11, wherein the breakable linear portion is an annular breakable linear portion.

14. The medical supply package according to claim 11, wherein:
the breakable linear portion is an annular breakable linear portion; and
a portion surrounded by the annular breakable linear portion is able to be cut off from the medical supply package.

15. A medical supply package for storing a medical supply, the medical supply package comprising:
a first sheet member; and
a second sheet member configured to face the first sheet member; wherein:
the first sheet member and the second sheet member are configured to store the medical supply therebetween;
the first sheet member and/or the second sheet member comprises a storage recess having a shape corresponding to an outer shape of the medical supply in a central portion;
the first sheet member and the second sheet member comprise:
abutting portions surrounding an outer periphery of the storage recess and configured to face each other,
an annular seal portion formed on the abutting portions and surrounding and hermetically sealing the storage recess, and
a non-seal portion provided on an outer side of the annular seal portion; and
at least one of the first sheet member and the second sheet member comprises a linear or band-shaped breakable portion, the breakable portion comprising:
an opening start portion including a start end in the non-seal portion,
a peelable portion continuous from the opening start portion and crossing a part of the annular seal portion, and
a breakable portion main part continuous from the peelable portion and crossing the storage recess, wherein the breakable portion does not inhibit airtightness of the annular seal portion.
